(19) United States
(12) Patent Application Publication
(10) Pub. No.: US 2025/0135128 A1
(43) Pub. Date: May 1, 2025

(54) COMBINATION VALVE AND FILTRATION UNIT

(71) Applicant: KILLARA I. P. PTY LTD, Dandenong South, Victoria (AU)

(72) Inventor: Mark Verschuur, Bayswater, Victoria (AU)

(21) Appl. No.: 18/695,132

(22) PCT Filed: Aug. 25, 2021
(86) PCT No.: PCT/AU2021/000047

§ 371 Date: Mar. 25, 2024

(51) Int. Cl. A61M 13/00; A61M 39/22

(52) U.S. Cl. CPC A61M 13/003; A61M 39/22; A61M 2205/75

(57) ABSTRACT

An in-line, combination valve and filtration unit to regulate and filter the exhaust flow of insufflation gas during laparoscopic surgery. The unit includes two light, thin, stiff, outwardly expanded shell parts of a more or less circular planform; filtration media being captured between abutting edges of the shell parts shaped for the purpose; an inlet shell part in the form of a simple shell; an outlet shell part in the form of a simple shell or incorporating an adjustable valve assembly; and panels and flow-deflecting elements fixed to the interiors of the shell parts to support the filtration media and/or direct gas flow through the shell parts. The shell parts have axially- or radially-arranged inlet and outlet fittings. The fittings taking the form of barb fittings, Luer fittings or the like.

This invention relates generally to apparatus employed in the performance of laparoscopic surgery. It relates specifically to means to regulate and control the release of insufflation gas from body cavities during laparoscopic surgery and to remove entrained matter from the gas outflow.

In the performance of laparoscopic surgery in the thorax and abdomen, insufflation is commonly employed. Insufflation, in this usage, is the pressurisation of a body cavity with a gas, the most commonly used of which is carbon dioxide. In the thorax, insufflation may be employed to create a surgical pneumothorax. In the abdomen, it is employed to provide working space between the internal organs and the abdominal wall. In all cases, access to the body cavity is gained via a trocar inserted through a suitably positioned incision. Suitable ducting and flow control means are provided at the proximal end of the trocar to perform and maintain insufflation. A separate incision may be provided to accommodate a separate trocar for the introduction of insufflation gas to a body cavity or the exhausting of insufflation gas from it. Surgery is performed by passing instruments through a trocar into the body cavity, suitable valve means being provided in the head part of the trocar to ensure that insufflation pressure is not lost.

Electrosurgery is frequently employed during laparoscopic surgery to cut, coagulate and dessicate tissue. Devices for this purpose take several forms, including heated probes for electrocautery, high-frequency (radio frequency) devices for the making of precise cuts with limited blood loss, and laser devices used for a number of surgical applications. The emissions generated by electrosurgery have been studied by the National Institute of Occupational Safety and Health division of the Center for Disease Control, USA. The studies have confirmed that the emissions can contain toxic gases and vapours such as benzene, hydrogen cyanide and formaldehyde, bioaerosols, dead and live cellular material (including blood fragments) and viruses. While smoke must be removed to prevent its obscuring the operating field, there is a clear risk to the health of medical personnel if emissions are simply vented into the operating theatre. It is therefore desirable that emissions generated by electrosurgery be promptly withdrawn from the body cavity and diverted, captured or processed in a way to minimise the possibility of injury to medical personnel.

Many methods have been developed for the withdrawal and processing of smoke and fumes generated by electrosurgery. Possibly the simplest is that exemplified by the Integral Insufflation Valve taught by Rockrohr in CA 2696493. In this device, a selectively rotatable, hollow valve handle has formed at its inner end moveable valve means which cooperate with fixed, complementary valve means formed in the head part of a trocar. Displacement of the handle part brings apertures of the fixed and moveable valve means into varying degrees of registration, permitting a flow of insufflation medium into or from the trocar via the hollow handle. Obviously, any discharge of smoke or fumes from the handle will represent a health danger to medical personnel working in proximity to it.

The Smoke Reducing Device for Minimally Invasive Surgery taught by Williams in U.S. Pat. No. 5,709,675 includes a housing sized to fit through a trocar opening leading to a body cavity, a filter being positioned within the housing between inlet and outlet openings, an air flow generator being located within the housing and positioned to draw air in through the inlet opening, through the filter, and exhausting it through the outlet opening, thereby drawing any smoke created during minimally invasive surgery through the filter; the smoke reducing device optionally forming part of an electrocautery device, allowing smoke created during minimally invasive surgery to be filtered internally of the body cavity. In this invention, the airflow generator takes the form of a fan driven by a remote drive motor via a flexible cable. The limited diameter of the housing would ensure the need for a fan of a diameter so small as to render it incapable of compelling a flow through the filter.

In the Automatic Smoke Evacuator System for a Surgical Laser Apparatus and Method Therefore taught by Cosmescu in U.S. Pat. No. 5,199,944, a laser laparoscope is provided with a conduit and fitting to provide CO₂ gas through the laparoscope for distension purposes, the laser laparoscope being coupled to a special trocar provided with exhaust holes near the tip (inside the body cavity), said exhaust holes being connected via conduit to an external fitting (outside the body cavity) to allow a vacuum source to draw smoke and CO₂ gas out of the body cavity, the smoke evacuator system optionally using either a built-in vacuum pump or an external vacuum source such as that typically provided in an operating room, electronics being provided which, upon detecting the activation of the cutting laser beam of the laser laparoscope, activate the smoke evacuation system, the system having the advantages of not requiring an addition incision and not requiring additional personnel to operate the system. The conduit of this invention adds unnecessarily to the diameter of the trocar and is inappropriate in the disposable, polymer trocars now in common use.

In the Disposable Laparoscopic Smoke Evacuation System taught by Trudel et al in U.S. Pat. No. 6,544,210, a small fan unit and filter are housed in a disposable housing adapted to be connected between two laparoscopic surgical instrument assemblies via tubing and powered by batteries or available AC power. It is doubtful if a fan unit is capable of properly compelling a flow of air through a suitable filter, particularly when battery-powered. The provision of a fan and drive motor is inappropriate comploutlety in a disposable item.

Similar comments in relation to comploutlety in disposable items might be made in relation to the Valleylab Laparoscopic Smoke Evacuation System supplied by Medtronic, Inc of Boulder, CO, USA; and of the Laservac Smoke Evacuation Systems supplied by I C Medical, Inc of Phoenix, AZ, USA.

In the Improvements Relating to Systems for Laparoscopic Surgery taught by Harwood et al in AU 2012101083, an insufflation system supplies a stream of gases at controlled temperature and humidity into the patient's abdominal or peritoneal cavity, a smoke evacuation system used in conjunction with the insufflation system withdrawing contaminated insufflation gas via a stopcock, discharge limb and filter, the stopcock and an optional adjustable outlet port on the filter being used to control flow; the filter comprising a hard shell enclosing suitable filter media, flow upstream of the filter passing via a pendent, breathable discharge limb in which condensed water vapour is allowed to collect and through the walls of which it passes to ambient. The system is complex, the necessary length and pendent form of the discharge limb is undesirable in the confines of the operating theatre and the greater part of the system is not adapted to the current practice of disposability

The Laparoscopic Smoke Evacuation System taught by Greff et al in U.S. Pat. No. 5,575,000 includes a trocar having a working channel adapted to provide access to an operative site and a stopcock communicating with the working channel, a source of vacuum being coupled through a fluid conduit to the stopcock to remove the smoke, filtration being provided along the fluid conduit to remove undesirable contaminants, the residual gas being exhausted to the room or the source of vacuum; a liquid collector is disposable along the fluid conduit together with valve means mechanically or electrically operable to control application of suction to the trocar. Similarly, the system is unnecessarily complex and is not adapted to the current practice of disposability.

The Laser Smoke Evacuation System and Method taught by Goodson et al in U.S. Pat. No. 4,735,603 includes a CO₂ gas pump connected through a control valve, a pressure sensor, and a bacterial filter to a laparoscopic tube inserted into the patient, a return line from a second laparoscopic tube in the patient through a smoke filter, a pressure sensor, a control valve, and a fluid trap into the return of the pump, and an insufflator connected into the patient to supply CO₂ gas lost by leakage and tissue absorption and to provide required distention of the patient cavity. Similarly, the system is complex and not adapted to the current practice of disposability.

The Wick and Relief Valve for Disposable Laparoscopic Smoke Evacuation System taught by Dean et al in US 2010/0094200 includes a hydrophilic wick positioned within the inlet system of the smoke device for absorbing moisture and trapping surgical waste entering the smoke evacuation device and a multi-outlet valve inserted into the outlet system of the smoke evacuation device to enable quick depressurization of the surgical site, flow being impelled from the inlet to the multi-outlet valve by a battery-powered fan. The invention is not adapted to the current practice of disposability.

The Filter Cartridge With Internal Gaseous Seal for Multimodal Surgical Gas Delivery System Having a Smoke Evacuation Mode taught by Mastri et al in U.S. Pat. No. 9,387,295 comprises a gas delivery device having a housing with a port for receiving insufflating gas from a gas source, a pump assembly for circulating pressurized gas throughout the system and a disposable gas conditioning unit or cartridge configured for operative association with the gas delivery device; the gas conditioning system including a first internal flow path for receiving pressurized gas delivered from the pump, a second internal flow path for delivering insufflating gas to the abdominal cavity at a desired flow rate and pressure and for facilitating periodic static pressure measurements from the abdominal cavity and a third internal flow path for returning pressurized gas to the pump; the first internal flow path including a nozzle assembly configured to accelerate the pressurized gas delivered by the pump and thereby generate a continuous pressure barrier contained within the gas conditioning unit, the pressure barrier or working zone that inhibits the egress of insufflating gas from the abdominal cavity functions to maintain a stable pneumoperitoneum; the housing of the gas conditioning unit including a vacuum chamber located within the third internal flow path, the vacuum chamber communicating with the nozzle chamber through a plurality of gas transfer ports to permit spent gas from the nozzle assembly to return to the pump for repressurization and circulation, a third filter element being disposed within the vacuum chamber for filtering gas returning to the pump from the patient's abdominal cavity. The invention is unnecessarily complex and not adapted to the current practice of disposability.

The Attachment for Removal of Smoke in Laparoscopic Surgery taught by Divilio et al in U.S. Pat. No. 5,417,655 includes a laparoscopic assembly and vacuum pump connected by the attachment, the attachment comprising a rigid hollow envelope having a plurality of apertures located toward a forward or upstream region of the envelope, the apertures preferably in a substantially concentric arrangement to allow ingress of air from the surrounding atmosphere in a substantially symmetrical flow, the hollow envelope 2 containing a first opening located at a forward (upstream) region and a second opening located at a rearward (downstream) region, a fluid flow tube disposed within the envelope being in a sealing arrangement with the envelope at the first opening, the tube extending into the interior of the hollow envelope to a rearward or downstream region thereof such that the flow of smoke from a patient cavity does not outlet the hollow envelope via the apertures but flows through the fluid flow tube of the device to the vacuum pump, the length of the fluid flow tube being at least as long as the distance from the inlet opening of the hollow envelope to a point immediately downstream of the apertures, the components preferably being integrally moulded out of plastic so as to make the cost sufficiently low that the assembly can be disposable. While simple and disposable, the attachment is apparently intended only to dilute smoke and fumes exhausted from a trocar without filtration.

The Fluid Flow Regulator for a Smoke Evacuation System taught by Wortrich et al in U.S. Pat. No. 6,592,543 comprises flow regulating means and conduit connecting means incorporating filtration means, the flow regulating means in one embodiment including a diaphragm and one or more orifices of predetermined diameter to provide a significant obstruction to the flow of fluid through the evacuation system, the fluid flow regulator making possible the continuous evacuation of surgical smoke while simultaneously maintaining the pneumoperitoneum in a distended position for the duration of the laparoscopic procedure; the regulator additionally allowing continual evacuation of surgical smoke without deflating the pneumoperitoneum. While the apparatus might be adaptable to disposability and it provides filtration and permits adjustment of flow rates by the interpositioning of orifices of different sizes, it is not unitary and therefore inconvenient to manage in the operating theatre environment.

The Variable Flow Smoke Evacuation Apparatus taught by Galley in GB 2524755 comprises a filter apparatus incorporating an integral variable flow control mechanism, a housing with an inlet and an outlet between which is arranged a filter, a structure defining a flow channel between the inlet and the outlet is mounted within the housing and a tube movable relative to the housing is coupled to the inlet or outlet, preferably by a screw thread; a closure member is located either on the structure or on an internal end of the tube such that the tube can be moved between a fully open position permitting through flow and a closed position in which it seats against the structure, intermediate positions throttling the rate of fluid flow; a smoke evacuation catheter is attached to the external end of the tube and rotation of the housing is used to adjust the flow rate, markings on the outside of the housing may indicate the relative venting rate associated with any particular position.

The Smoke Filter for Laparoscopic Surgery taught by Mo et al in CN210727855 comprises a shell with an (air inlet) opening in the top and an air outlet in the bottom, a flow adjusting mechanism arranged at the air outlet and a filtering layer; a control structure being rotationally connected to an opening of the shell and extending into an inner cavity of the shell to be connected with the flow adjusting mechanism, the mechanism being adjusted by rotating the control structure; the filter layer being located at an outlet of the through (flow) hole (duct); gas entering via the gas inlet sequentially passing through the through the hole (through flow duct), the filtering layer and the flow adjusting mechanism and finally discharging from the gas outlet; the smoke filter being used in conjunction with existing equipment. The smoke filter is adapted to disposability, but the dies for its manufacture in polymer materials are complex and its compact arrangement limit the filtration area available and the provision for capturing condensed water vapour.

The following patents are indicative of the state of the art of smoke evacuation during laparoscopic surgery:
KR20140147405 (Lee et al);
JP2018192267 (Yue-Sing);
CN209752512 (Gu);
CN209713173 (Qiu);
CN209285591 (Luo et al);
CN209172489 (Tan et al);
CN2091153955 (Zheng et al);
CN2089926629 (Zhao);
CN208822987 (Yang et al);
CN208212086 (Zhu et al);
CN204092190 (Yin et al);
CN110403692 (Chen et al).

The object of the present invention is to provide a device to intercept insufflation gas exhausted from the human body cavity during laparoscopic electrosurgery; which is compact, unitary in its assembled form and of relatively simple construction, requiring relatively simple dies to mould its components from polymer materials; which is modular in arrangement, readily assembled and adapted to be disposable; which incorporates means to regulate the flow of gas that are easily managed in the operating theatre; and which has efficient gas flow through filtration means able to remove water vapour, chemical compounds, fumes and smoke and entrained particulates.

According to the present invention, a combination valve and filtration unit comprises suitable filtration media captured at its edges between two light, thin, stiff, outwardly expanded inlet and outlet shell parts moulded from a suitable polymer material, the abutting edges of the shell parts being sealingly joined by the engagement of complementary joining shapes moulded into them or by other means. The inlet shell part incorporates an axially-arranged inlet fitting while the outlet shell part incorporates an outwardly-directed, cylindrical valve housing, the axis of which is made collinear with that of the shell parts and inlet fitting, a radially-arranged outlet fitting being formed on the exterior of the valve housing, its bore communicating with the interior of the housing. Rotationally and sealingly accommodated within the cylindrical valve housing is a cylindrical valving element provided with a valving slot of tapering width or a plurality of valving apertures of increasing diameter, the slot or apertures being situated in a plane normal to the rotational axis of the valving element and coincident with the axis of the outlet fitting; parts of the tapered valving slot or valving apertures of varying diameters being able to be brought into registration with the bore of the outlet fitting, thereby regulating the volume flow through the outlet fitting and, thereby, through the filtration media. A knob formed on its exterior surface is gripped to position the valving element appropriately. A pointing indicator is preferably formed on the knob and complementary graduations formed on the exterior surface of the cylindrical valve housing to provide a reference of knob position. Preferably, stops are provided to limit the rotational travel of the valving element. Also preferably, a detent is provided at pre-determined flow value positions of the valving element, a complementary projection entering each detent with an audible or haptic ‘click’; the detents being formed in the inner surface of the cylindrical valve housing and the projection on the outer surface of the valving element, or vice versa. The filtration media include types or combinations of types for capture of bacteria and, smoke and fumes, entrained solids and condensed water vapour. The inlet shell part is optionally enlarged to accommodate wicking and water storage media to immobilise and store captured condensed water vapour. A separate module to capture and store condensed water vapour is optionally fixed to the inlet side of the present invention.

The various aspects of the present invention will be more readily understood by reference to the following description of preferred embodiments given in relation to the accompanying drawings in which:

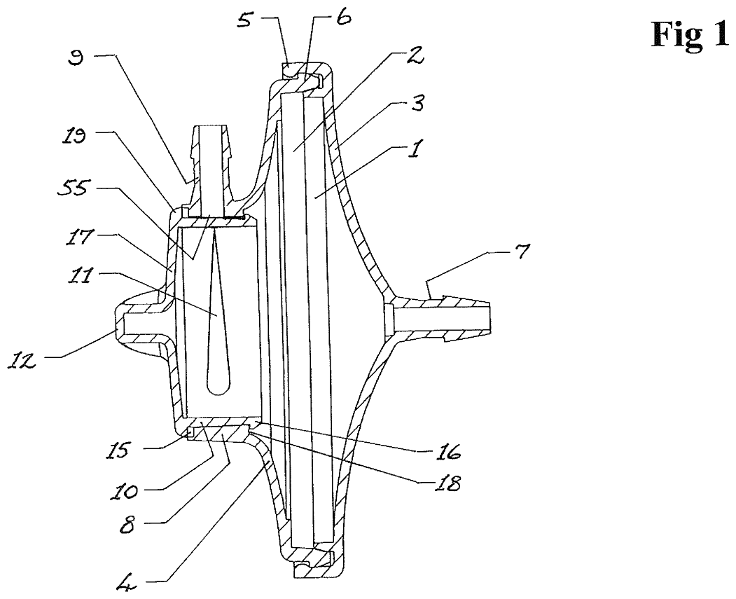

FIG. 1 is a longitudinal cross-sectional view on a plane passing through the centrelines of the inlet and outlet fittings of a first embodiment of the present invention;

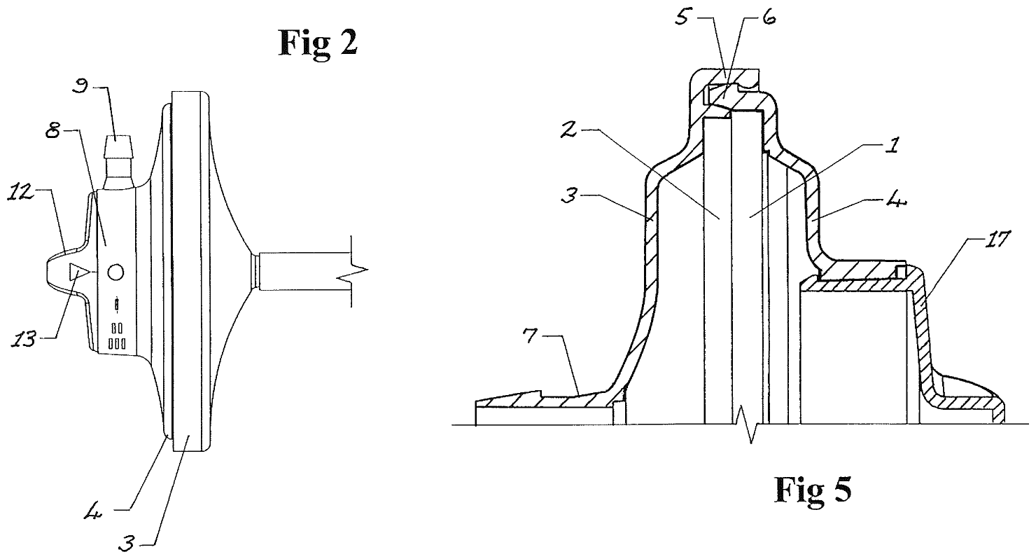

FIG. 2 is a side view of the invention of FIG. 1;

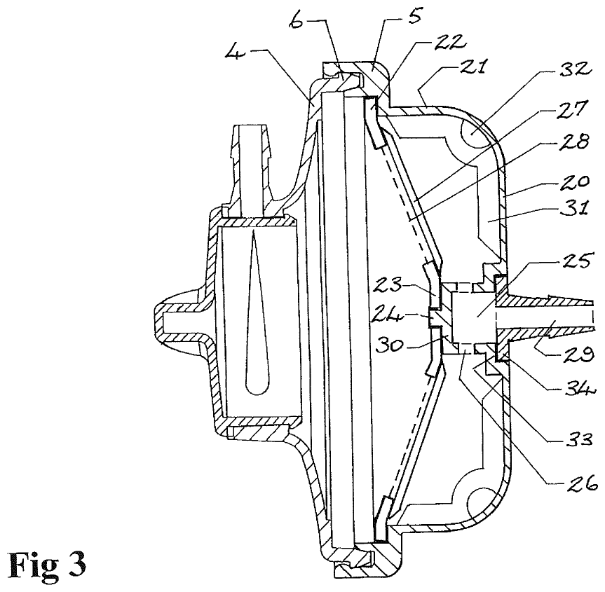

FIG. 3 is a longitudinal cross-sectional view on the same plane as FIG. 1 of an alternative embodiment of the present invention in which the shell part on the inlet side is enlarged to accommodate provisions to capture condensed water vapour;

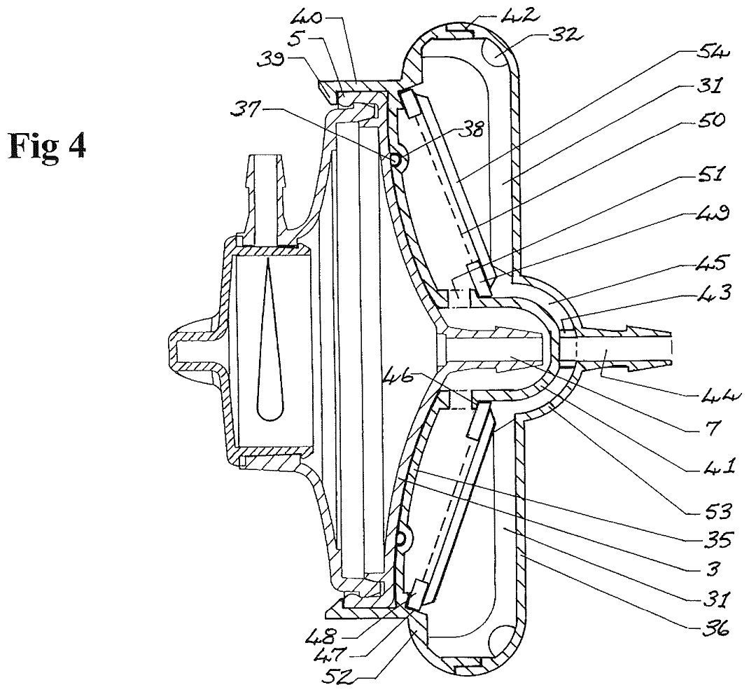

FIG. 4 is a longitudinal cross-sectional view on the same plane as FIG. 1 of a further alternative embodiment of the present invention in which a separate module to capture condensed water vapour is sealingly attached to the shell part on the inlet side;

FIG. 5 is a partial longitudinal cross-sectional view on the same plane as FIG. 1 of an alternative embodiment of the invention of FIG. 1;

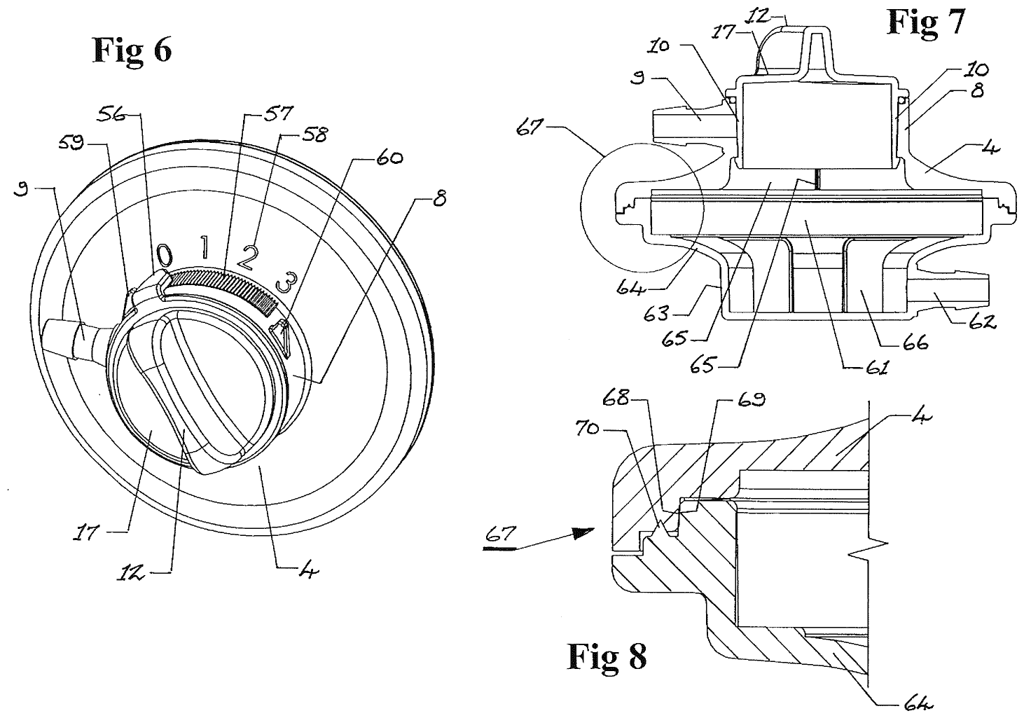

FIG. 6 is an oblique, face view of the present invention depicting alternative means to adjustably fix positions of the valving element;

FIG. 7 is a transverse cross-sectional view of the present invention depicting an alternative location of the inlet fitting;

FIG. 8 is a fragmentary transverse cross-sectional view of an alternative method of joining the shell parts of the present invention, the area depicted being circled in FIG. 7;

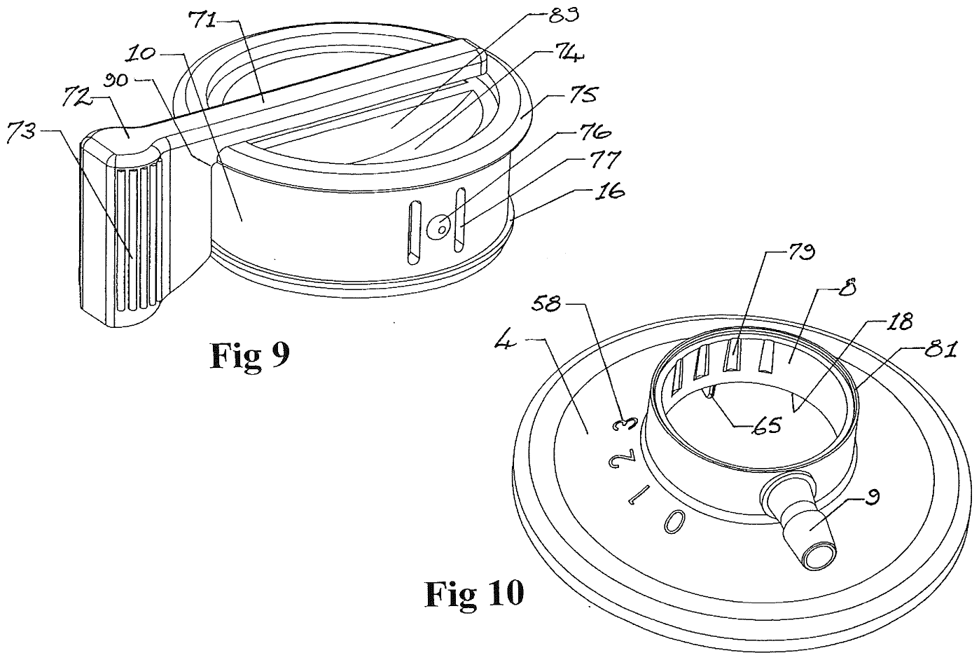

FIG. 9 is an oblique view of an alternative embodiment of the valving element of the present invention in which the valve position adjusting knob is replaced with a thumb lever;

FIG. 10 is an oblique view of an alternative embodiment of the outlet shell part adapted to support the valving element of FIG. 9;

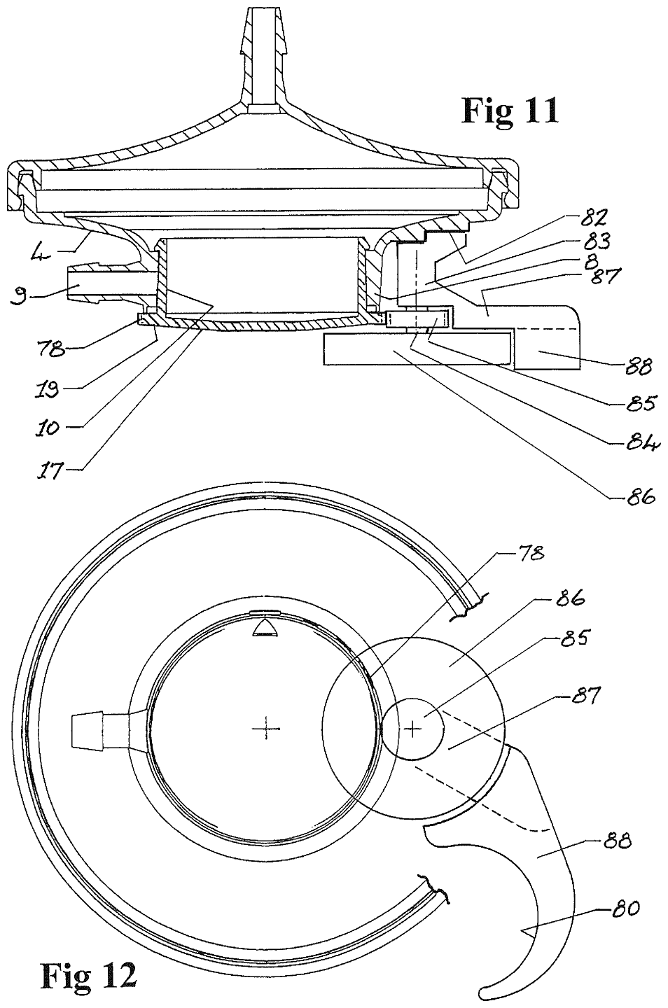

FIG. 11 is a longitudinal cross-sectional view on a plane passing through the centrelines of the inlet and outlet fittings in which the valve position is adjusted with a thumb wheel;

FIG. 12 is a partially schematic face view of the embodiment of FIG. 11;

FIG. 13 is a side view of an alternative embodiment of the inlet shell part;

FIG. 14 is a transverse cross-sectional view of the embodiment of FIG. 13;

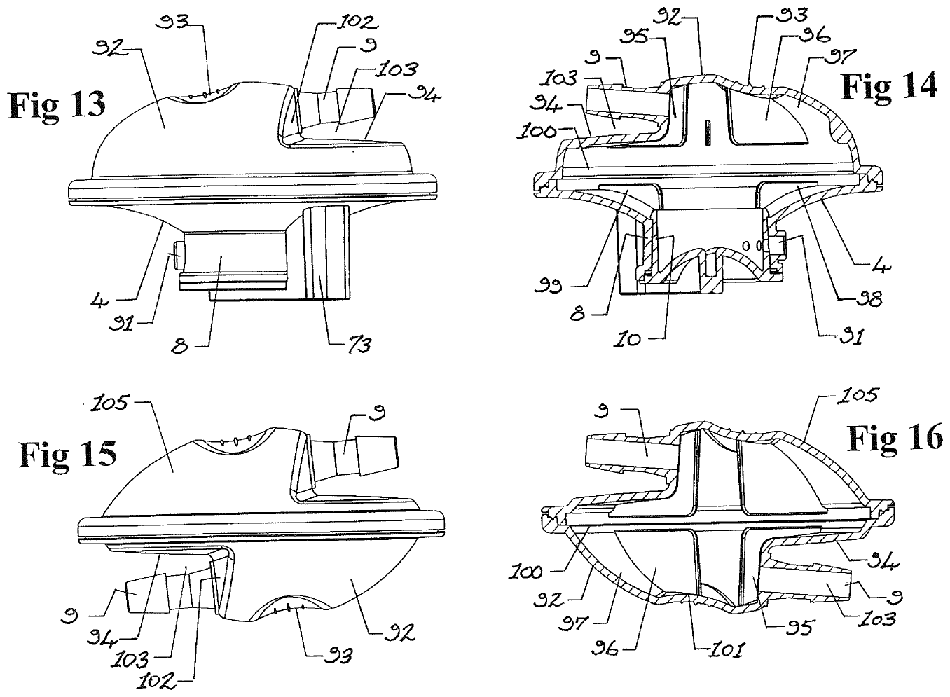

FIG. 15 is a side view of alternative embodiment in which the outlet shell part is a replication of the inlet shell part, the two shell parts being fixed together separated by 180 degrees;

FIG. 16 is a transverse cross-sectional view of the embodiment of FIG. 15;

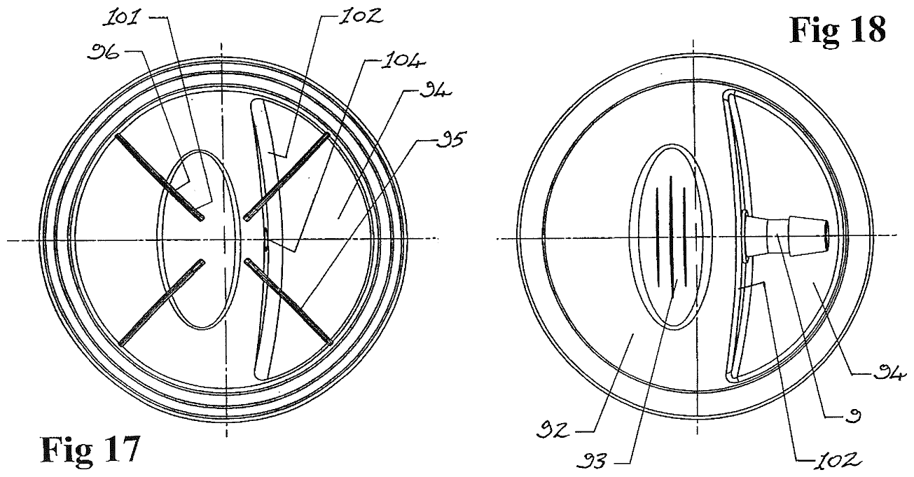

FIG. 17 is a face view of the interior of the inlet shell part of FIGS. 13 to 16;

FIG. 18 is a face view of the exterior of the inlet shell part of FIGS. 13 to 16;

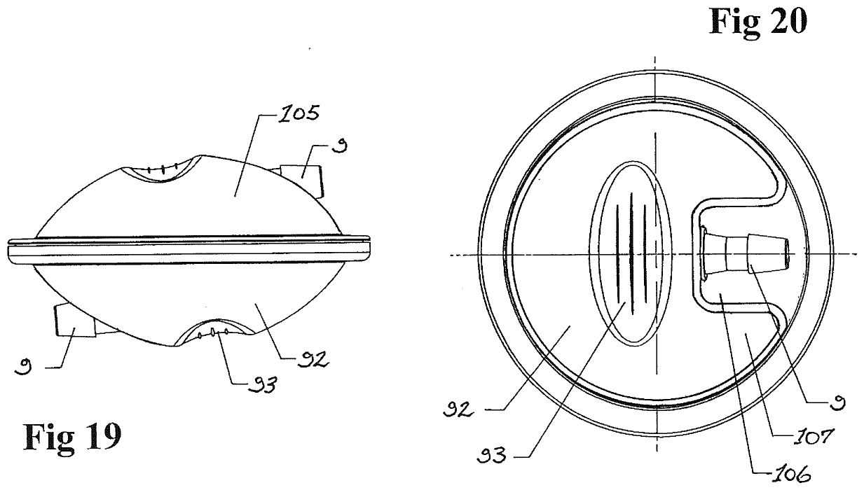

FIG. 19 is a side view of an alternative embodiment in which the inlet shell part and, optionally, the outlet shell parts is extended such that the inlet and outlet fitting of each is enclosed within a recess;

FIG. 20 is a face view of the exterior of the inlet shell part of FIG. 19;

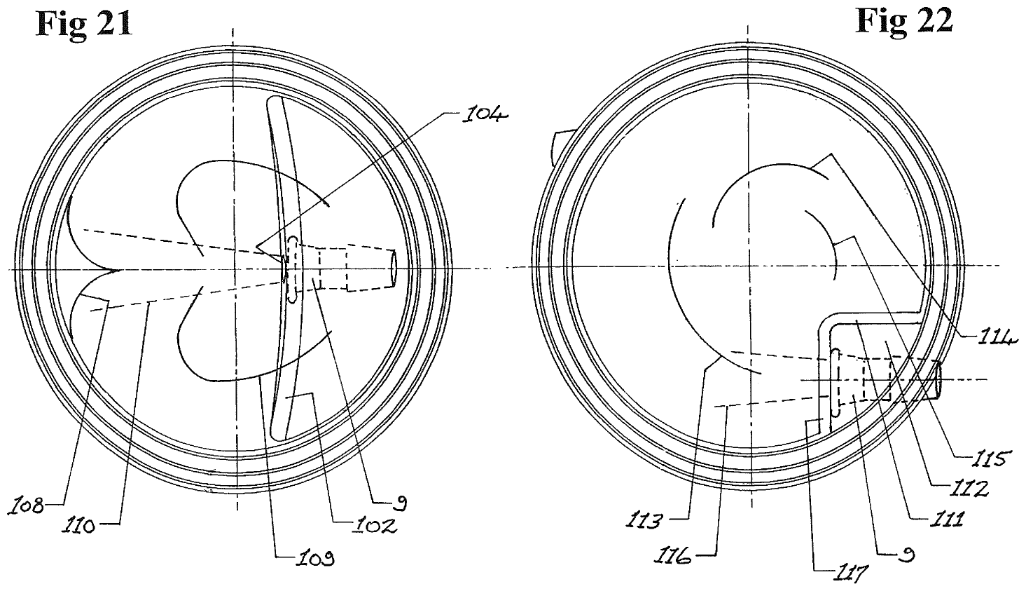

FIG. 21 is a schematic face view of the interior of an alternative embodiment of the inlet shell part of FIG. 18;

FIG. 22 is a schematic face view of the interior of another alternative embodiment of the inlet shell part of FIG. 18 in which the inlet fitting is disposed more or less tangentially.

With reference to FIGS. 1 and 2, an in-line, combination valve and filtration unit is provided to filter and regulate the exhaust flow of insufflation gas during laparoscopic surgery. Said combination valve and filtration unit comprises two light, thin, stiff, outwardly expanded shell parts 3, 4 of more or less circular planform preferably moulded from a suitable polymer material having a thickness in the range 1.5 to 3.0 millimetres, the abutting edges of said shell parts being sealingly joined by the engagement of complementary joining shapes 5, 6 or by welding or bonding. Suitable filtration media 1, 2 is captured between said shell parts by its edges being sealingly sandwiched between edge parts of said shell parts shaped for the purpose. Inlet shell part 3 incorporates a radially- or axially-arranged inlet fitting 7, outlet shell part 4 incorporating an outwardly-directed, cylindrical valve housing 8, the axis of which is made collinear with that of said shell parts and said inlet fitting, a radially-arranged outlet fitting 9 formed on the exterior of said valve housing communicating with its interior. Rotationally and sealingly accommodated within said valve housing is valving element 10 provided with a valving slot 11 of tapering width or a plurality of valving apertures (not shown) of increasing diameter, said slot or said apertures being situated in a plane normal to the rotational axis of said valving element and coincident with the axis of said outlet fitting; parts of said tapered valving slot or valving apertures of varying diameters being able to be brought into registration with said outlet fitting, thereby regulating the volume of gas flowing through said outlet shell and, thereby, through said filtration media. Accommodated within circumferential space 15 between circumferential flange 19 of valving element closure 17 of said valving element and the outer end surface of said valve housing is narrow wave spring (not shown) which acts to maintain flange 16 formed on the inner end of said valving element in rotational, sealing contact with shoulder 18 formed at the inner end of said valve housing. A centrally located knob 12 formed on the exterior surface of valving element closure 17 is gripped to position said valving element appropriately. A pointing indicator 13 is formed on said knob and complementary graduations (typical graduations depicted as 14) are formed on the exterior surface of the cylindrical valve housing to provide a visual reference of the position of said knob. In the preferred embodiment, stops (not shown) are provided to limit the rotational travel of the valving element. Also in the preferred embodiment, an indexing detent (not shown) is provided at pre-determined flow value positions of said valving element, a complementary indexing projection (not shown) entering each said indexing detent with an audible or haptic ‘click’, said indexing detents being formed in the inner surface of said cylindrical valve housing and said indexing projection on the outer surface of said valving element, or vice versa. Said inlet and outlet fittings take the form of barb fittings, Luer fittings or the like.

In the preferred embodiment, filtration media 1 takes the form of a finely-divided, granulated or combination of finely-divided and granulated, activated carbon suitable for vapour phase applications and in sheet form. In a first embodiment, said activated carbon is bound into a solid form using a suitable binder in the form of a thermosetting resin, such as phenolic resin (subsequently carbonised), or a thermoplastic, such as polyethylene homopolymer and/or copolymer. In another embodiment, said activated carbon is immobilised in a suitable matrix in the form of a sheet of bound cellulosic or polymer fibres, or a sheet of an open-cell, microcellular or macrocellular foamed polymer. Said foamed polymers include, but are not limited to, low-density polyethylene, polyethylene, polyimide, polypropylene, polystyrene, polyurethane, polyvinyl chloride and silicone, or any combination of these. In another embodiment, said activated carbon is sandwiched between layers of needle-punched or non-woven fabric. In another embodiment, said activated carbon takes the form of felt, cloth or fabric made from activated carbon fibre. In another embodiment, said activated carbon is ‘doped’ or mixed with elements or compounds, such as magnesium dioxide, copper oxide, copper sulphate, lead acetate, zinc acetate, phosphoric acid, metallic silver, or tris-(hydroxymethyl) aminomethane to improve the capture or neutralisation of pollutants such as carbon monoxide, ethylene oxide, hydrogen sulphide, mercaptans, ammonia, amines, ozone, formaldehyde, other aldehydes or bacteria. Depending upon its form, said activated carbon is optionally supported from a circumferential frame (not shown). Also in the preferred embodiment, filtration media 2 takes the form of a sheet of non-woven, fabric melt blown from polyester, polypropylene, polystyrene, polyurethane, polyamide, polyethylene, or polycarbonate or any combination of these. Said filtration media is generally of the type typically employed in the making of medical face masks meeting standards EN 14683, ASTM F2100, EN 1822 and ISO 29463 and is supported from a circumferential frame (not shown). In the preferred embodiment, said filtration media is able to capture entrained viruses, bacteria, liquid droplets and the like down to a diameter of approximately 0.1 micron. In an alternative embodiment (not shown), said filtration media is sandwiched with and located on the outlet side of said activated carbon sheet. In another alternative embodiment (not shown), a sheet of finely woven, polymer gauze treated to be superhydrophobic is provided on the inlet side of said activated carbon sheet, said superhydrophobic polymer gauze preventing a substantial component of entrained, condensed water vapour from entering said activated carbon sheet and, thereby, blocking gas flow through it. In another alternative embodiment, said filtration media is moulded onto one or both sides of a substantially rigid grille woven from a suitable wire or injection moulded from a suitable polymer, the aperture size of said grille falling in the range 2.0 to 6.0 millimetres, said grille acting to support said filtration media.

With reference to FIG. 3, said inlet shell part incorporates optional water interception and storage means. In this embodiment, inlet shell part 20 is developed axially to create a shallow cylindrical form 21 coaxial with said outlet shell part and closed by a closure panel 20 disposed parallel to the axis of joining of said shell parts. Edge joining shape 5 is extended axially to accommodate the additional thickness of the circumferential frame of a water capture screen, the screen capturing water droplets entrained in said inlet air flow. Formed in the centre of said inlet shell part is inwardly-projecting short duct 25, the circumferential walls of which are provided with a plurality of apertures 26, the open outer end of said short duct supporting inlet fitting 29, flange 34 of which is bonded or welded into complementary recess 33. Button 24 is formed on the outer surface of closure 30 of said short duct. Said water capture screen assembly has a shallow, conical form and comprises circumferential frame 22, inner frame 23, a plurality of rigid, radially-arranged connecting bars 27 joining said frames, and screen 28. Said screen takes the form of a sheet of finely woven, polymer gauze treated to be superhydrophobic and fixed to said circumferential frame and said inner frame. Circumferential frame 22 is sealingly captured between the edge parts of shell parts 20, 4, inner frame 23 being sealingly fixed to said inner surface of closure 30 of said short duct by the seating of an aperture formed in said frame over button 24. A suitable sealant is applied as required to circumferential frame 22 and to inner frame 23. The inner surface of shell part 20 is substantially lined with a layer of a strongly wicking material 31, said material covering a strip of a strongly water-absorbent material 32 located more or less at the point of greatest width between said inlet shell part and said screen. In the preferred embodiment, said wicking material is made from a microfiber product generally comprising split conjugated fibres of polyester and polyamide, said water-absorbent material being sodium polyacrylate. In use, a flow of gas entering at inlet fitting 29 passes into short duct 25 and thence via apertures 26 to screen 28. Owing to the superhydrophobic nature of said screen, condensed water vapour droplets accumulate on said screen and, having grown to a size exceeding 1.5 to 2.0 millimetres, detach and fall to and accumulate in the lowest zone of that part of said inlet shell part bounded by said screen. In normal use, said zone will be adjacent said circumferential frame. Said wicking material extends into said zone, carrying away said water by capillary action to said water-absorbent material. Said water absorbent material is located in said widest effective point to allow for its swelling as it absorbs water.

With reference to FIG. 4, a discrete module having a shallow cylindrical form is optionally fixed to the exterior of said inlet shell part to capture and store condensed water vapour. Said module comprises two thin, stiff shell parts, inner part 35 and outer part 36, preferably moulded from a suitable polymer material. Said inner part is shaped to closely conform to the outer surface of inlet shell part 3 and is sealed to it by soft ‘O’ ring 37 accommodated in groove 38. Said module is fixed to the invention of FIG. 1 by a plurality of elastic fingers 40, each of which terminates in a sprag 39 which engages the square edge of joining shape 5. Said inner part is made with a centrally-located, axially-projecting dome 41 that covers inlet fitting 7. A plurality of apertures 51 provided at the base of said axially projecting dome permit a flow of gas from said module to inlet fitting 7. Inner part 35 is joined by bonding or welding to outer part 36 around circumferential zone 42, said parts preferably being thickened at this point and a half lap or other suitable arrangement being provided to achieve a sound joint. Said outer part is made with a broad, flat area 36 disposed more or less parallel to the plane of joining of said inlet and outlet shell parts, an axially-located, outwardly-projecting dome 53 formed on said flat panel covering dome 41. Axially-located inlet fitting 44 is formed on dome 53, an inward flow of gas being free to pass via space 45 between said domes and into said module. Short struts 43 are formed on the inner surface of dome 53 and, in the preferred embodiment, their inner ends are bonded to dome 41, connecting said domes together, said short struts not impeding the flow of gas into space 45. The inner part 35 of said module is thickened at point 52 and a pad 47 provided for attachment of circumferential frame 48 of a water capture screen. Dome 41 is thickened at its inner end to create shoulder 46 to locate inner frame 23 of said water capture screen. Said water capture screen assembly comprises circumferential frame 48, inner frame 49, a plurality of rigid, radially-arranged connecting bars 54 joining said frames, and screen 50. Said screen takes the form of a sheet of finely woven, polymer gauze treated to be superhydrophobic and fixed to said circumferential frame and said inner frame. In the preferred embodiment, said circumferential frame is bonded or welded to pad 47 and said inner frame is bonded or welded to dome 41. In the preferred embodiment, the inner surface of module shell 36 on the inlet side of said water capture screen is substantially lined with a layer of a strongly wicking material 31, said material covering a strip of a strongly water-absorbent material 32 located more or less at the widest effective point of said module on the inlet side of said screen. In the preferred embodiment, said wicking material is made from a microfiber product generally comprising split conjugated fibres of polyester and polyamide, said water-absorbent material being sodium polyacrylate. In use, a flow of gas entering at inlet fitting 44 passes between short struts 43 and via space 45 between said domes to screen 50. Owing to the superhydrophobic nature of said screen, condensed water vapour droplets accumulate on said screen and, having grown to a size exceeding 1.5 to 2.0 millimetres, detach and fall to and accumulate in the lowest zone of that part of said inlet shell part. In normal use, this zone will be adjacent said circumferential frame. Said wicking material extends into said zone, carrying away said water by capillary action to said water-absorbent material. Said water absorbent material is located in said widest effective point of said inlet shell part to allow for its swelling as it absorbs water.

With reference to FIG. 5, in another alternative embodiment, those parts of shell parts 3, 4 immediately adjacent their edge parts and between which filtration media 1, 2 are captured are bulged outwardly and the generality of said shell parts is shaped to be substantially parallel to said filtration media, thereby facilitating a flow of gas through the outer parts of said filtration media. In a further alternative embodiment (not shown), suitable flow deflecting panels are formed on the inner surface of inlet shell part 3 to more evenly distribute the flow of gas from inlet fitting 7 over said filtration media.

With reference to FIG. 6, in an alternative embodiment, knob 12 is formed on closure 17 of said valving element. Indexing arm 56 is formed on the outer edge of said closure, extending inwardly and positioned closely to and parallel to the exterior surface of cylindrical valve housing 8. Said indexing arm is provided centrally along its inner surface with a narrow, elongated, raised indexing projection (not shown) approximately triangular in cross-sectional shape and having an angle of approximately 60 degrees between its two exposed sides. Said indexing projection engages a circumferential band 57 of suitable serrations formed on the exterior surface of said cylindrical valve housing. In a further alternative embodiment, said serrations are replaced by a series of spaced-apart, parallel grooves engaged by said indexing projection. Said engagement of said indexing projection with said serrations or said grooves acts to positively retain said valving element in any selected position, stops 59, 60 limiting travel of said indexing arm around said cylindrical valve housing and, thereby, limiting the range of rotational displacement of said valving element. Numbering 58 formed on the surface of shell part 4 around the base of said cylindrical valve housing provide a reference for the position of said valving element.

With reference to FIG. 7, inlet shell part 64 has formed on it a centrally-located, outwardly orientated, closed cylindrical projection 63, said cylindrical projection having radially-located inlet fitting 62 formed on it. In the preferred embodiment, the axis of said inlet fitting is orientated 180 degrees from that of outlet fitting 9. In alternative embodiments, the axis of said inlet fitting is orientated at any angle of separation from that of said outlet fitting. In the preferred embodiment, a plurality of panels formed on or fixed to the inner surface of the closed end of said cylindrical projection extend inwardly, their free ends abutting the surface of filtration media 61, thereby supporting said filtration media against displacement by fluid pressure. Also in the preferred embodiment, a plurality of narrow bars 65 in a cruciform or star-shaped arrangement is formed on the interior surface of cylindrical valve housing 8 beneath valving element 10, the inner edges of said bars being parallel to and abutting the surface of filtration media 61, thereby supporting said filtration media against displacement by fluid pressure. In an alternative embodiment (not shown), cylindrical projection 63 is rotationally and sealingly supported in a suitable bearing arrangement provided centrally in inlet shell part 64, the orientation of the axis of said inlet fitting thereby being made universally variable. In another alternative embodiment (not shown), a plurality of circumferentially-arranged detents is provided on the exterior surface of inlet shell part 64 around the base of rotationally-supported, cylindrical projection 63 at pre-determined angular positions of said inlet fitting, a short arm projecting radially from the base of said cylindrical projection having a complementary projecting element which enters each said detent with an audible or haptic ‘click’ as said cylindrical projection is rotationally displaced. Engagement of said projecting element with said detents acts to positively retain said cylindrical projection in any selected position.

With additional reference to FIG. 8, in an alternative embodiment, inlet and outlet shell parts 4, 64 are joined at their peripheries by ultra-sonic welding. In this embodiment, during said welding process said shell parts are positively located one to another by abutment of complementary locating faces 68, 69. An energy director 70 is provided on one of the two surfaces to be joined, said energy director passing around the whole of the intended joint surface. Said energy director is triangular in cross-sectional shape with an angle of approximately 60 degrees between its two exposed sides. The two said shell parts are depicted in the figure in a position shortly after commencement of the welding process.

With reference to FIGS. 9 and 10, in an alternative embodiment, closure 74 of valving element 10 is made substantially concave and thumb lever 72 is fixed to the free end of a diametrically-arranged bar 71 formed on said closure. Said bar is connected to said concave part of said closure by web 89, said concavity facilitating the gripping of said bar by the fingers of a user. Said thumb lever is provided on either side with coarse texturing 73 to facilitate its gripping by a gloved finger or thumb. Said valving element is rotationally and sealingly accommodated within cylindrical valve housing 8 formed axially on outlet shell part 4, being retained within said cylindrical valve housing by flange 16 formed on the inner end of said valving element in rotational, sealing contact with shoulder 18 formed at the inner end of said cylindrical valve housing. As described in relation to FIG. 1, said valving element is provided with a valving slot (not shown) of tapering width or a plurality of valving apertures (not shown) of increasing diameter, said slot or said apertures being situated in a plane normal to the rotational axis of said valving element and coincident with the axis of an outlet fitting (not shown); parts of said tapered valving slot or valving apertures of varying diameters being able to be brought into registration with said outlet fitting, thereby regulating the volume flow of gas through said valve unit. Positions of said valving element are defined by rounded button 76 formed on the exterior surface of said valving element indexing with a plurality of regularly-spaced recesses 79 formed in the interior surface of said cylindrical valve housing. Said recesses are made elongated in an axial sense and extend to the free edge of said cylindrical valve housing to facilitate release from the die during moulding. Axially-arranged slots 77 are provided in the wall of said valving element close to and to either side of said rounded button, said slots facilitating the inwards, elastic displacement of said button as it passes between said recesses, an audible and haptic ‘click’ being generated as said button enters each said recess. In the preferred embodiment, during rotational displacement of said valving element, a lug (not shown) formed on the underside of outer flange 75 of said valving element traverses an arcuate cut-away zone (not shown) formed in the free edge 81 of said cylindrical valve housing, the abutment of said lug with the ends of said cut-away zone limiting displacement of said valving element. In an alternative embodiment, an edge 90 at the junction of said thumb lever with transverse bar 71 replaces said lug and traverses said cut-away zone. A pointer (not shown) of the type depicted as 13 in FIG. 2 is optionally provided at the free end of said transverse bar, indicating the position of said valving element with reference to position-identifying elements 58 formed on the exterior surface of outlet shell part 4.

With reference to FIGS. 11 and 12, valving element positioning knob 12 (as depicted in FIG. 1) is deleted and edge 19 of closure 17 of valving element 10 is extended radially and its circumferential edge formed into gear band 78. Outwardly-projecting pillar 83 is bonded or welded to suitable attachment pads 82 formed on the exterior surface of outlet shell part 4, shaft 84 being rotationally supported in a suitable bearing formed in said pillar. Fixed to the outer end of said shaft is serrated thumb wheel 86 and fixed to said shaft between the inner face of said thumb wheel and the end surface of said pillar is gear wheel 85. Said gear wheel engages gear band 78 such that rotation of said thumb wheel causes rotation of said valving element within cylindrical valve housing 8. In an alternative embodiment (not shown), a friction washer is provided between gear wheel 85 and the end surface of pillar 83, said washer acting to prevent uncontrolled rotation of said thumb wheel and said gear wheel. In the preferred embodiment, the diameter of gear wheel 85 is made to be one quarter of that of gear band 78 such that one turn of thumb wheel 86 produces a 90 degree rotational displacement of valving element 10. In alternative embodiments the diameter of said gear wheel is made greater or smaller in relation to that of said gear band. The peripheral surface of said thumb wheel is coarsely textured to facilitate its engagement by a gloved thumb or finger. Also supported from said pillar is radially-projecting arm 87, the outer end of said arm supporting curved finger hold 88. In operation, the passing of the index finger around curved part 80 of said finger hold whilst applying pressure to said thumb wheel allows an operator to positively control the position of said valve unit while adjusting the position of said valving element.

With reference to FIGS. 13 and 14, inlet shell part 92 is substantially expanded to create an approximately part-spherical shape, thereby improving the distribution of the flow of gas within said shell part and over filtration media 1, 2 (as depicted in FIGS. 1 and 2). Recess 103 is created by cutting away from said expanded inlet shell part an area in the form of a circular segment having a width in the range 0.25 to 0.4 of the radius of said shell part and to partial depth of said shell part, said recess being defined by two intersecting panels, said panels being flat or slightly curved, a first panel 102 more or less defining a chord and a second panel 94 orientated more or less parallel to the plane of attachment of said shell parts; inlet fitting 9 being situated diametrically in said recess and fixed to said first panel over an inlet aperture (depicted as 104 in FIG. 17). Outlet shell part 4 and adjustable valve assembly are substantially as described in relation to FIG. 7. In the preferred embodiment, ribbed, shallow depression 93 is provided situated more or less centrally on the exterior of said inlet shell part to facilitate gripping. In an alternative embodiment (not shown), said ribbed area is flat or is slightly raised. Four radially-arranged, flat panels 95, 96 are provided within said expanded inlet shell part separated by 90 degrees and bonded or welded to its interior surface, the free, inner edges of said panels being coplanar, in operation abutting the inlet side surface of and supporting said filtration media. Open area 97 is provided between the free, side edges of panels 96 and the interior surface of said expanded inlet shell part to facilitate the circulation of gases within said shell part. Four radially-arranged, flat panels 98, 99 are provided within outlet shell part 4 bonded or welded to its interior surface, the free, inner edges of said panels being coplanar, in operation abutting the outlet side surface of and supporting said filtration media. Gap 100 created between the free, inner edges of panels 95, 96 and 98, 99 is made in an appropriate width to snugly accommodate said filtration media. Stub 91 is provided on the exterior of valve housing 8 for the attachment of a suitable outlet fitting.

With reference to FIGS. 15 and 16, in an alternative embodiment, inlet shell part 92 is made as described in relation to FIGS. 13 and 14, while outlet shell part 105 is made essentially as a mirror image of said inlet shell part and is fixed to said inlet shell part but orientated 180 degrees in relation to it.

With reference to FIGS. 17 and 18, in the embodiments of FIGS. 14 and 15 and FIGS. 15 and 16, radially-arranged panels 95, 96 separated by 90 degrees are provided within expanded inlet shell parts 92. In the preferred embodiment, zones 101 of said panels immediately adjacent their most central parts are bonded or welded to the interior surfaces of said shell parts. Recess 103 (as depicted in FIGS. 13-16) is created by cutting away part of said expanded inlet shell part and inserting transverse panels 102 and 94 orientated more or less normal to each other. Inlet fitting 9 is situated diametrically in said recess and is fixed to transverse panel 102 over inlet aperture 104. In the preferred embodiment, the inner edges of said radially-arranged panels are positioned and shaped such that a flow of gas entering said inlet fitting passes via aperture 104 to the interior of said inlet shell part and impinges upon the inner edges of said panels, causing it to be deflected more or less evenly into the three spaces downstream of said aperture including to part of the zone beneath transverse panel 94. In an alternative embodiment (not shown), suitable apertures are provided in radially-arranged panels 95 to permit a flow of gas into the zone beneath transverse panel 94.

With reference to FIGS. 19 and 20, inlet shell part 92 is further expanded by the creation of expanded extensions 107, thereby creating small recess 106 within which inlet fitting 9 is diametrically mounted; outlet shell part 105 optionally being further expanded in the same way.

With reference to FIG. 21, radially-arranged panels 95, 96 (as depicted in FIG. 17) are deleted and expanded inlet shell part 92 (as depicted in FIG. 18) is provided with curved, symmetrically-arranged flow-deflecting elements (approximate positions indicated by lines 108, 109). Said flow deflecting elements are made from a suitable thin, stiff material having a thickness in the range 1.5 to 2.5 millimetres and bonded or welded to the inner surface of said inlet shell part. Flow-deflecting elements 109, located more or less centrally within said inlet shell part and closer to said inlet aperture, act to partially intercept a flow of gas (indicated in broken line as 110) entering via inlet fitting 9 and aperture 104 and deflect it to zones on either side of said inlet shell part and to zones either side of and beneath said inlet fitting. Flow-deflecting elements 108 fixed to the end wall of said inlet shell part (opposed to said inlet fitting) and centred on the extended axis of said inlet fitting act to deflect the remainder of said flow of gas to zones on either side of said inlet shell part remote from said inlet aperture; said flow-directing elements acting to provide a more or less even distribution of gas throughout the whole interior of said inlet shell part. In an alternative embodiment (not shown), a narrow slot is provided at the apex of flow deflecting elements 108 to permit a flow of gas to the interior of said flow deflecting elements.

With reference to FIG. 22, inlet shell part 92 is further expanded to create a more or less circular planform, with the exception of a small recess 112 defined by two intersecting, short panels 111, 117, inlet fitting 9 being tangentially mounted over an inlet aperture (not shown) on panel 117 within said recess. The interior of said inlet shell part is provided with a series of curved flow-deflecting elements (approximate positions indicated by lines 113, 114, 115), said elements being made from a suitable thin, stiff material having a thickness in the range 1.5 to 2.5 millimetres and bonded or welded to the inner surface of said inlet shell part. Said flow-deflecting elements are spaced apart in a radial sense with their ends overlapping, each successive element being smaller and closer to the centre of said inlet shell part. A flow of gas (indicated in broken line as 116) entering via inlet fitting 9 is partially intercepted by the largest said flow-deflecting element and then sequentially deflected by successive said flow-deflecting elements around the central zones of said inlet shell part and zones closer to said inlet fitting, the remainder of said flow of gas being deflected by the internal wall of said inlet shell part around zones of said inlet shell part most remote from said inlet fitting. Said process of interception and deflection act to provide a more or less even distribution of gas throughout the whole interior of said inlet shell part.

In an alternative embodiment (not shown), said inlet and outlet fittings are fixed to their parent components using a ball and socket arrangement, said ball and socket arrangement permitting the alignment of said inlet fittings to be readily adjusted.

The fact that components normally mating are depicted to different scales is of no significance.

The present invention should be taken to include all feasible combinations of the features described herein.

1-36. (canceled)

37. An in-line, combination flow valve and filtration unit to regulate and filter the exhaust flow of insufflation gas during laparoscopic surgery; said unit comprising a housing formed by the abutment of two complementary, outwardly-expanded, circular or approximately circular, light, thin, stiff shell parts, one said shell part being an inlet shell part and incorporating an inlet flow fitting, the other said shell part being an outlet shell part and incorporating an outlet flow fitting, filtration media being captured between said shell parts; said outlet shell part incorporating an adjustable valve assembly by means of which the rate of outflow of said insufflation gas through said filtration media is regulated; panels fixed to the interiors of said shell parts supporting said filtration media and acting to deflect the flow of said insufflation gas evenly towards all parts of said filtration media; said adjustable valve assembly comprising an outwardly-directed, cylindrical valve housing formed on said outlet shell part, the axis of which is made collinear with that of said inlet and outlet shell parts; a cylindrical valving element turned by a knob and rotationally and sealingly accommodated within said valve housing; a radially-arranged outlet fitting formed on the exterior of said valve housing and communicating with its interior; a valving slot of tapering width or a plurality of valving apertures of increasing diameter formed in said valving element, said slot or said apertures being situated in a plane situated normal to the rotational axis of said valving element and coincident with the axis of said outlet fitting; in use, said tapered valving slot or valving apertures of varying diameters being able to be brought into registration with said outlet fitting, thereby regulating the volume of said insufflations gas flowing through said outlet shell part and, thereby, through said filtration media.

38. The combination valve and filtration unit of claim 37 wherein a pointing indicator is formed on said valving element knob and complementary graduations are formed on the exterior surface of said cylindrical valve housing or said outlet shell part to provide a visual reference of the position of said knob; and/or in which stops are provided to limit the rotational travel of said valving element.

39. The combination valve and filtration unit of claim 37 wherein the abutting edges of said shell parts are sealingly joined by the engagement of complementary joining shapes moulded into them or by ultra-sonic welding, an energy director being moulded into one of the two surfaces to be joined; shaping of those parts of said injection moulding dies for the forming of said complementary joining shapes being facilitated by the round planform shape of said shell parts.

40. The combination valve and filtration unit of claim 37 wherein said inlet shell part is developed axially to create a shallow cylindrical form coaxial with said outlet shell part and closed by a closure panel disposed parallel to the axis of joining of said shell parts, said shell parts being sealingly joined by engagement of complementary joining shapes formed on their abutting edges; a water capture screen assembly of shallow, conical form is accommodated within said cylindrical form, said assembly incorporating a sheet of finely woven, polymer gauze treated to be superhydrophobic and fixed to a circumferential frame sealingly captured between said shell parts and to an inner frame at its apex sealingly supported from the inner surface of said closure panel; the inner surface of said shallow, cylindrical form being substantially lined with a layer of a strongly wicking material covering a strip of a strongly water-absorbent material.

41. The combination valve and filtration unit of claim 37 wherein a discrete module having a shallow cylindrical form has one face shaped to be complementary with the exterior shaping of said inlet shell part and is sealingly fixed to the exterior of said inlet shell part, retained in place by means of claws engaging the edge of said inlet shell part, a dome of said module covering and supplying gas flow to said inlet fitting, another axially-mounted inlet fitting being provided on the outer surface of the outer panel of said module; a water capture screen assembly of shallow, conical form being accommodated within said module, said assembly incorporating a sheet of finely woven, polymer gauze treated to be superhydrophobic and fixed to a circumferential frame which, in turn, is sealingly fixed to the inner surface of said module immediately adjacent the outer edge of said inlet shell part and to an inner frame sealingly fixed to said dome; said module being made in two parts to facilitate installation of said water capture screen assembly; the inner surface of said shallow, cylindrical form being substantially lined with a layer of a strongly wicking material covering a strip of a strongly water-absorbent material.

42. The combination valve and filtration unit of claim 41 wherein said wicking material is made from a microfiber product generally comprising split conjugated fibres of polyester and polyamide, said water-absorbent material taking the form of sodium polyacrylate; in use, condensed water vapour droplets accumulating on said superhydrophobic screen, being absorbed by said wicking material and carried by capillary action to said water-absorbent material.

43. The combination valve and filtration unit of claim 37 wherein an indexing arm is formed on said knob extending inwardly and positioned close to and parallel to the exterior surface of said valve housing, said indexing arm being provided centrally along its inner surface with a narrow, elongated, raised indexing projection approximately triangular in cross-sectional shape and having an angle of approximately 60 degrees between its two exposed sides, said indexing projection engaging a circumferential band of suitable serrations formed on the exterior surface of said valve housing, engagement of said indexing projection with said serrations acting to positively retain said valving element in any selected position; and, wherein said serrations are replaced by a series of spaced-apart, parallel grooves engaged by said indexing projection, engagement of said indexing projection with said grooves acting to positively retain said valving element in any selected position.

44. The combination valve and filtration unit of claim 37 wherein said inlet shell part has formed on it an axially-located, outwardly orientated, closed cylindrical projection, said cylindrical projection having a radially-located inlet fitting formed on it, a plurality of panels formed on or fixed to the inner surface of the closed end of said cylindrical projection extend inwardly, their free ends abutting the inlet side surface of said filtration media; the inner edges of a plurality of narrow bars in a cruciform or star-shaped arrangement formed on the interior surface of said valve housing inwardly of said valving element abutting the outlet side surface of said filtration media; the abutment of said panel free ends and said inner edges of said narrow bars against said surfaces of said filtration media acting to support said filtration media against displacement by fluid pressure; and wherein said cylindrical projection is rotationally and sealingly supported in a centrally-located, suitable bearing arrangement, the orientation of the axis of said inlet fitting fixed to said cylindrical projection being thereby being made universally variable.

45. The combination valve and filtration unit of claim 44 wherein a plurality of circumferentially-arranged detents is provided on the exterior surface of said inlet shell part around the base of said cylindrical projection at pre-determined angular positions of said inlet fitting, a short arm projecting radially from the base of said cylindrical projection having a complementary projecting element which enters each said detent with an audible or haptic ‘click’ as said cylindrical projection is rotationally displaced; engagement of said projecting element with said detents acting to positively retain said cylindrical projection in any selected position.

46. The combination valve and filtration unit of claim 37 wherein the outer end of said valving element is formed into a closure made substantially concave, a thumb lever being fixed to the free end of a bar formed diametrically on said closure and extending across its diameter, said transverse bar being connected to said concave part of said closure by a web, said concavity facilitating the gripping of said bar; said thumb lever being provided on either side with coarse texturing to facilitate its gripping by a gloved finger or thumb.

47. The combination valve and filtration unit of claim 46 wherein positions of said valving element are defined by a rounded button formed on the exterior surface of said valving element indexing with a plurality of regularly-spaced recesses formed in the interior surface of said cylindrical valve housing, axially-arranged slots being provided in the wall of said valving element close to and to either side of said rounded button to facilitate the inwards, elastic displacement of said button as it passes between said recesses, an audible and haptic ‘click’ being generated as said button enters each said recess.

48. The combination valve and filtration unit of claim 37 wherein a circumferential gear band is formed on the closure of said valving element and a serrated thumb wheel is rotationally supported from said outlet shell part, a gear wheel coupled to said thumb wheel engaging said gear band such that rotation of said thumb wheel causes rotation of said valving element within said valve housing, a curved finger hold supported from said outlet shell part allowing an operator to positively control the position of said valve unit while rotating said thumb wheel to adjust the position of said valving element.

49. The combination valve and filtration unit of claim 37 wherein said inlet shell part is substantially expanded to create an approximately spherical shape, thereby improving the distribution of the flow of gas within said shell part and over said filtration media; a recess being created in said expanded inlet shell part by cutting away an area in the form of a circular segment having a width in the range 0.25 to 0.4 of the radius of said shell part and to partial depth of said shell part, said recess being defined by two intersecting panels which are flat or slightly curved, a first panel orientated to approximate a chord of the previously defined spherical shape and a second panel orientated approximately parallel to the plane of attachment of said shell parts; said inlet fitting being situated over an inlet aperture on a diameter passing through the centre of said first panel; and, wherein a ribbed area is provided situated approximately centrally on the exterior of said inlet shell part to facilitate gripping, said ribbed area taking the form of a shallow depression, a flat area or a slightly raised area.

50. The combination valve and filtration unit of claim 49 wherein four radially-arranged panels are provided within said expanded inlet shell part separated by 90 degrees and bonded or welded to its interior surface, the free, innermost edges of said panels being coplanar; an open area being provided between the free, side edges of panels opposed to said inlet fitting and the interior surface of said expanded inlet shell part to facilitate the circulation of gases within said shell part; four radially-arranged panels being provided within said outlet shell part separated by 90 degrees and bonded or welded to its interior surface, the free, innermost edges of said panels being coplanar; a gap created between said free, innermost edges of said panels in said inlet shell part and said outlet shell part being made in an appropriate width to snugly accommodate, and thereby support, said filtration media.

51. The combination valve and filtration unit of claim 49 wherein said inlet shell part and said outlet shell part are both substantially expanded to create approximately spherical shapes; a recess being created in both said expanded shell parts by cutting away areas in the form of circular segments having widths in the range 0.25 to 0.4 of the radius of said shell parts and to the partial depth of said shell parts, said recesses each being defined by two intersecting panels which are flat or slightly curved, a first panel orientated to approximate a chord of the previously defined spherical shape and a second panel orientated approximately parallel to the plane of attachment of said shell parts; said inlet fitting being fixed to said first panel over an inlet aperture on a diameter passing through the centre of said recess; and said expanded shell parts being fixed together with one rotated 180 degrees in relation to the other.

52. The combination valve and filtration unit of claim 50 wherein:
said inlet shell part is substantially expanded to create an approximately spherical shape, thereby improving the distribution of the flow of gas within said shell part and over said filtration media; a recess being created in said expanded inlet shell part by cutting away an area in the form of a circular segment having a width in the range 0.25 to 0.4 of the radius of said shell part and to partial depth of said shell part, said recess being defined by two intersecting panels which are flat or slightly curved, a first panel orientated to approximate a chord of the previously defined spherical shape and a second panel orientated approximately parallel to the plane of attachment of said shell parts; said inlet fitting being situated over an inlet aperture on a diameter passing through the centre of said first panel; and, wherein a ribbed area is provided situated approximately centrally on the exterior of said inlet shell part to facilitate gripping, said ribbed area taking the form of a shallow depression, a flat area or a slightly raised area, and
the inner edges of said radially-arranged panels in said inlet shell part are positioned and shaped such that a flow of gas entering the interior of said inlet shell part via said inlet fitting and said inlet aperture is partially intercepted by said panel inner edges, causing part of said flow to be deflected into the three spaces downstream of said inlet fitting and aperture defined by said panels, including to part of the zone beneath said recess-defining second panel, suitable apertures being provided in said radially-arranged panels adjacent said inlet fitting and aperture to permit a flow of gas into the zone beneath said recess-defining second panel.

53. The combination valve and filtration unit of claim 51 wherein said inlet shell part is further expanded by the creation of expanded extensions to either side of said inlet fitting, thereby creating a small recess sufficient to accommodate said inlet fitting, said inlet fitting being fixed over an inlet aperture on a diameter passing through the centre of said recess; and, wherein said outlet shell part is further expanded by the creation of expanded extensions to either side of said inlet fitting, thereby creating a small recess sufficient to accommodate said inlet fitting, said inlet fitting being fixed over an inlet aperture on a diameter passing through the centre of said recess; said outlet shell part being fixed to said inlet shell part but rotated 180 degrees in relation to it.

54. The combination valve and filtration unit of claim 49 wherein the interior of said expanded inlet shell part is provided with curved, symmetrically-arranged flow-deflecting elements, said flow-deflecting elements being made from a suitable thin, stiff material having a thickness in the range 1.5 to 2.5 millimetres and bonded or welded to the inner surface of said inlet shell part; a first pair of said flow-deflecting elements approximately centrally-located within said inlet shell part acting to partially intercept a flow of gas entering via said inlet aperture and deflect it to zones on either side of said inlet shell part and to zones beneath and to either side of said inlet fitting; a second pair of said flow-deflecting elements being fixed to the wall of said inlet shell part, centred on the extended axis of said inlet fitting and more distant from said inlet aperture, acting to deflect the remainder of said flow of gas to zones on either side of said inlet shell part remote from said inlet aperture; said flow-deflecting elements acting to provide an approximately even distribution of gas throughout the whole interior of said inlet shell part.

55. The combination valve and filtration unit of claim 49 wherein said inlet shell part is further expanded to create an approximately circular planform, with the exception of a small recess defined by two intersecting, short panels, said inlet fitting being tangentially mounted over an inlet aperture on one said panel within said recess; the interior of said expanded inlet shell part being provided with a series of curved, flow-deflecting elements, said flow-deflecting elements being made from a suitable thin, stiff material having a thickness in the range 1.5 to 2.5 millimetres and bonded or welded to the inner surface of said inlet shell part; said flow-deflecting elements being spaced apart in a radial sense with their ends overlapping, each successive element being progressively shorter and closer to the centre of said inlet shell part; a flow of gas entering said inlet shell part via said inlet fitting being partially intercepted by the largest said flow-deflecting element and then sequentially deflected by said series of flow-deflecting elements around central zones of said inlet shell part and zones closer to said inlet fitting; the remainder of said flow of gas being deflected by the internal wall of said inlet shell part around zones of said inlet shell part most remote from said inlet fitting; said process of interception and deflection acting to provide an approximately even distribution of gas throughout the whole interior of said inlet shell part.

56. The combination valve and filtration unit of claim 37 wherein said inlet flow fitting and said outlet flow fitting are fixed, respectively to said inlet shell part and said outlet shell part using ball and socket arrangements, said ball and socket arrangements permitting the alignment of said inlet and outlet flow fittings to be adjusted relative to a respective central, longitudinal axis of either an inlet shell part or an outlet shell part.
